(19) United States
(12) Patent Application Publication
(10) Pub. No.: US 2021/0260039 A1
(43) Pub. Date: Aug. 26, 2021

(54) NUTRITIONAL COMPOSITIONS FOR ENHANCEMENT OF MUSCLE PERFORMANCE

(71) Applicant: MEND NUTRITION INC., New York, NY (US)

(72) Inventors: Eziah SYED, New York, NY (US); Hans DRYER, Eugene, OR (US); Erin C. OWEN, Eugene, OR (US)

(73) Assignee: MEND NUTRITION INC., New York, NY (US)

(21) Appl. No.: 17/261,713

(22) PCT Filed: Jul. 19, 2019
(86) PCT No.: PCT/US19/42616

§ 371 Date: Jan. 20, 2021

Related U.S. Application Data
(60) Provisional application No. 62/701,072, filed on Jul. 20, 2018.

(51) Int. Cl. A61K 31/4172; A23L 33/175; A61K 31/198; A61K 31/205; A61K 31/405; A61K 9/00; A61P 21/06

(52) U.S. Cl. CPC A61K 31/4172; A23L 33/175; A61K 31/198; A61P 21/06; A61K 31/405; A61K 9/0095; A61K 31/205

(57) ABSTRACT

Compositions comprising a plurality of particular essential amino acids, and methods of enhancing muscle performance by administering such compositions are provided herein.

### REFERENCE TO GOVERNMENT GRANTS

This invention was made with government support under Grant No. AG046401 awarded by the National Institutes of Health. The government has certain rights in the invention.

### FIELD

The present disclosure generally relates to methods for alleviating distressed tissue of the human body, such as amelioration of post-surgery muscle atrophy, by administering nutritional compositions comprising a plurality of particular essential amino acids.

### BACKGROUND

Diseases of the joints, such as knee and thigh joints, occur due to trauma, arthritis, osteoarthritis and other such related conditions and can be painful and debilitating. Surgery to alleviate the pain commonly referred to as total knee arthroplasty (TKA) and total hip arthroplasty (THK) involve removing the damaged cartilage and bone from the surface of the knee joint or hip joint and replacing these tissues with artificial products such as metal and plastic. According to the Mayo Clinic an estimated 4.7 million Americans have undergone TKA and 2.5 million have undergone THA to date.

Several studies have shown that there is significant muscle atrophy in the regions of the joints surrounding the post-TKA and post-THA surgical procedures. This muscle atrophy can persist for years or even become permanent. The atrophy of muscles impairs the return of strength in the joints that have undergone TKA and THA surgical procedures which may affect the overall functional mobility of the individual who has undergone these procedures. Thus, for example, quadriceps atrophy and weakness jeopardize balance, reduce functional mobility, and increase the risk of falls. Currently, a primary means employed to regain at least some functionality and amelioration of muscle atrophy after TKA or THA surgery is physical therapy.

Amino acids have been employed as nutritional supplements, and attempts have been made to employ amino acids in formulations in efforts to promote human and veterinary health. In general, such attempts have involved relatively large amounts of amino acids to achieve desired beneficial results. Multiple attempts to use essential amino acid (EAA) mixtures to augment muscle growth, however, have yielded mixed results at best. In addition, because of difficulties associated with the highly disagreeable taste of some amino acids, preparation of amino acid compositions has been difficult. For example, compositions using methionine, as well as other amino acids, have been avoided for at least this reason. An additional difficulty with nutritional supplements for older adults is that older adults generally compensate for increased energy (i.e., calories) delivered by nutritional supplements by reducing food intake. It is, therefore, desirable to design a supplement that stimulates muscle synthesis more efficiently than food or common protein supplements. Thus, there continues to be a need for a nutritional or dietary supplement that can be easily administered to individuals in need of enhancing tissue, such as muscle, performance.

### SUMMARY

The present disclosure provides compositions comprising: about 30% (wt) to about 34% (wt) L-leucine, or a salt thereof; about 13% (wt) to about 17% (wt) L-lysine, or a salt thereof; about 11% (wt) to about 15% (wt) L-phenylalanine, or a salt thereof, about 7% (wt) to about 11% (wt) L-valine, or a salt thereof, about 6% (wt) to about 10% (wt) L-threonine, or a salt thereof; about 6% (wt) to about 10% (wt) L-isoleucine, or a salt thereof; about 6% (wt) to about 10% (wt) L-methionine, or a salt thereof, about 3% (wt) to about 7% (wt) L-histidine, or a salt thereof; and about 1% (wt) to about 4% (wt) L-tryptophan, or a salt thereof.

The present disclosure also provides methods of enhancing muscle performance in a human subject, the method comprising administering to the subject at least one serving of a composition comprising: about 30% (wt) to about 34% (wt) L-leucine, or a salt thereof, about 13% (wt) to about 17% (wt) L-lysine, or a salt thereof, about 11% (wt) to about 15% (wt) L-phenylalanine, or a salt thereof, about 7% (wt) to about 11% (wt) L-valine, or a salt thereof; about 6% (wt) to about 10% (wt) L-threonine, or a salt thereof, about 6% (wt) to about 10% (wt) L-isoleucine, or a salt thereof, about 6% (wt) to about 10% (wt) L-methionine, or a salt thereof, about 3% (wt) to about 7% (wt) L-histidine, or a salt thereof, and about 1% (wt) to about 4% (wt) L-tryptophan, or a salt thereof.

### DESCRIPTION OF EMBODIMENTS

The terminology used herein is for the purpose of describing particular embodiments only and is not intended to be limiting.

As used herein, the singular forms “a,” “an” and “the” include plural referents unless the context clearly dictates otherwise.

As used herein, the terms “subject” and “patient” are used interchangeably. A subject may include any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates. In some embodiments, the subject is a human.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the phrase “essential amino acid” refers to an amino acid that cannot be synthesized de novo by a subject, and therefore must be supplied by diet or supplement.

As used herein, the phrase “semi-essential amino acid” refers to an amino acid that is considered conditionally essential in the human diet, and its synthesis can be limited under special pathophysiological conditions such as severe catabolic distress.

As used herein, the phrase “non-standard amino acid” refers to a non-proteinogenic amino acid that occurs naturally in a cell.

As used herein, the term “attenuate” refers to a decrease or lessening of the symptoms or signs of the underlying disease or disorder.

As used herein, the term “ameliorate” refers to any reduction in the extent, severity, frequency, and/or likelihood of a symptom or clinical sign characteristic of a particular condition.

The present disclosure provides compositions comprising a mixture of essential amino acids (EAA) that are useful as described herein. In some embodiments, the compositions containing the EAA have increased amounts of lysine and, optionally, one or more additional amino acids. In some embodiments, one or more of the amino acids in the EAA compositions are in free form. In some embodiments, one or more of the amino acids in the EAA compositions are in a salt form.

In some embodiments, the compositions comprise a blend of amino acids containing at least 13% (wt) lysine in which the proportion of each amino acid is tailored to enhance muscle protein synthesis. Such compositions may also contain other EAA, semi-essential amino acids (e.g., conditional amino acids), non-essential amino acids, and their hydroxy analogs and keto analogs.

In some embodiments, the compositions comprise at least about 13% (wt) lysine. In some embodiments, the compositions comprise from about 13% (wt) to about 25% (wt) lysine, from about 13% (wt) to about 17% (wt) lysine, or from about 15% (wt) to about 15.5% (wt) lysine.

In some embodiments, the compositions comprise: L-leucine, or a salt thereof; L-lysine, or a salt thereof; L-phenylalanine, or a salt thereof; L-valine, or a salt thereof; L-threonine, or a salt thereof; L-isoleucine, or a salt thereof; L-methionine, or a salt thereof; L-histidine, or a salt thereof; and L-tryptophan, or a salt thereof.

In some embodiments, the concentrations of the amino acids may be in any proportion to enhance muscle protein synthesis. In some embodiments, the concentration of L-lysine, or salt thereof, in the composition is greater than 13% (wt) of the total amino acids present. In some embodiments, the concentration of L-lysine, or salt thereof, in the composition is from about 13% (wt) to about 25% (wt) by weight of the total amino acids present. In some embodiments, the concentration of L-leucine, or salt thereof, in the composition is from about 3% (wt) to about 40% (wt) of the total amino acids present. In some embodiments, the concentration of L-phenylalanine, or salt thereof, in the composition is from about 8% (wt) to about 20% (wt) of the total amino acids present. In some embodiments, the concentration of L-valine, or salt thereof, in the composition is from about 2% (wt) to about 16% (wt) of the total amino acids present. In some embodiments, the concentration of L-threonine, or salt thereof, in the composition is from about 2% (wt) to about 15% (wt) of the total amino acids present. In some embodiments, the concentration of L-isoleucine, or salt thereof, in the composition is from about 4% (wt) to about 12% (wt) of the total amino acids present. In some embodiments, the concentration of L-methionine, or salt thereof, in the composition is from about 4% (wt) to about 12% (wt) of the total amino acids present. In some embodiments, the concentration of L-histidine, or salt thereof, in the composition is from about 3% (wt) to about 9% (wt) of the total amino acids present. In some embodiments, the concentration of L-tryptophan, or salt thereof, in the composition is from about 0.5% (wt) to about 4% (wt) of the total amino acids present.

In some embodiments, the concentration of L-lysine, or salt thereof, in the composition is from about 13% (wt) to about 17% (wt) of the total amino acids present. In some embodiments, the concentration of L-leucine, or salt thereof, in the composition is from about 30% (wt) to about 34% (wt) of the total amino acids present. In some embodiments, the concentration of L-phenylalanine, or salt thereof, in the composition is from about 11% (wt) to about 15% (wt) of the total amino acids present. In some embodiments, the concentration of L-valine, or salt thereof, in the composition is from about 7% (wt) to about 11% (wt) of the total amino acids present. In some embodiments, the concentration of L-threonine, or salt thereof, in the composition is from about 6% (wt) to about 10% (wt) of the total amino acids present. In some embodiments, the concentration of L-isoleucine, or salt thereof, in the composition is from about 6% (wt) to about 10% (wt) of the total amino acids present. In some embodiments, the concentration of L-methionine, or salt thereof, in the composition is from about 6% (wt) to about 10% (wt) of the total amino acids present. In some embodiments, the concentration of L-histidine, or salt thereof, in the composition is from about 3% (wt) to about 7% (wt) of the total amino acids present. In some embodiments, the concentration of L-tryptophan, or salt thereof, in the composition is from about 1% (wt) to about 4% (wt) of the total amino acids present.

In some embodiments, the concentration of L-lysine, or salt thereof, in the composition is from about 15% (wt) to about 16% (wt) of the total amino acids present. In some embodiments, the concentration of L-leucine, or salt thereof, in the composition is from about 31% (wt) to about 32% (wt) of the total amino acids present. In some embodiments, the concentration of L-phenylalanine, or salt thereof, in the composition is from about 13% (wt) to about 14% (wt) of the total amino acids present. In some embodiments, the concentration of L-valine, or salt thereof, in the composition is from about 8% (wt) to about 9% (wt) of the total amino acids present. In some embodiments, the concentration of L-threonine, or salt thereof, in the composition is from about 8% (wt) to about 9% (wt) of the total amino acids present. In some embodiments, the concentration of L-isoleucine, or salt thereof, in the composition is from about 7% (wt) to about 8% (wt) of the total amino acids present. In some embodiments, the concentration of L-methionine, or salt thereof, in the composition is from about 7% (wt) to about 8% (wt) of the total amino acids present. In some embodiments, the concentration of L-histidine, or salt thereof, in the composition is from about 5% (wt) to about 6% (wt) of the total amino acids present. In some embodiments, the concentration of L-tryptophan, or salt thereof, in the composition is from about 2% (wt) to about 3% (wt) of the total amino acids present.

In some embodiments, the compositions comprise about 31.6% (wt) L-leucine, about 15.4% (wt) L-lysine, about 13.2% (wt) L-phenylalanine, about 8.9% (wt) L-valine, about 8.1% (wt) L-threonine, about 7.7% (wt) L-isoleucine, about 7.5% (wt) L-methionine, about 5.5% (wt) L-histidine, and about 2.1% (wt) L-tryptophan.

In some embodiments, the composition comprises about 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9 or about 25.0% (wt) lysine.

In some embodiments, a plurality of amino acids comprises one or more essential amino acids. In some embodiments, preferential use of essential amino acids compared to nonessential amino acids in a particular composition can reduce the amount of amino acids that are administered to elicit an effective response. Six amino acids are considered conditionally essential in the human diet: arginine, cysteine, glycine, glutamine, proline, and tyrosine.

In some embodiments, the composition comprises leucine, lysine, phenylalanine, valine, threonine, isoleucine, methionine, histidine, and tryptophan. In some embodiments, the composition comprises leucine, lysine, phenylalanine, valine, threonine, isoleucine, methionine, histidine, tryptophan, and a conditionally essential amino acid. When a composition comprises all nine essential amino acids and other amino acids, the nine essential amino acids are at least about 70% (wt) of the total amino acids present. In some embodiments, the nine essential amino acids are about 50% (wt) to about 100% (wt) of the total amino acids present. In some embodiments, the percentage of the total essential amino acids plus semi-essential amino acids or other amino acids, is about 5% (wt) to 50% (wt) of the total amino acids present.

In some embodiments, a plurality of amino acids comprises one or more nonstandard amino acids. Examples of nonstandard amino acids include, but are not limited to, L-carnitine, GABA, hydroxyproline, hydroxylysine, pyrrolysine, selenomethionine, hypusine, L-DOPA, 2-aminobutyric acid, dehydralanine, and gamma-carboxyglutamic acid. In some embodiments, the composition comprises phenylalanine, valine, threonine, tryptophan, methionine, leucine, isoleucine, lysine, histidine, and at least one non-standard amino acid.

In some embodiments, the compositions also comprise essential amino acids and other functional ingredients such as, but not limited to, probiotics and branched chain fatty acids.

In some embodiments, the compositions further comprise at least one nutrient chosen from an omega-3 fatty acid and/or biotin. In some embodiments, the compositions further comprise at least two nutrients chosen from an omega-3 fatty acid and/or biotin. The overall contribution of the one or more nutrients to the total weight of the compositions in such cases is substantially less than the contribution of the plurality of amino acids. Generally, the one or more nutrients comprise no more than about 10% (wt), no more than about 5% (wt), or no more than about 3% (wt) of the composition. In some embodiments, the one or more nutrient is biotin. In some embodiments, the one or more nutrient is one or more omega-3 fatty acids. In some embodiments, the one or more nutrient is biotin and one or more omega-3 fatty acids.

In some embodiments, the compositions comprise: i) leucine, from about 13% (wt) to about 15.9% (wt) lysine, phenylalanine, valine, threonine, isoleucine, methionine, histidine, and tryptophan; ii) at least one conditionally essential amino acid; and iii) at least one nonstandard amino acid. In some embodiments, the compositions comprise: i) leucine, from about 13% (wt) to about 15.9% (wt) lysine, phenylalanine, valine, threonine, isoleucine, methionine, histidine, and tryptophan; ii) at least one conditionally essential amino acid precursor; and iii) at least one non-standard amino acid. In some embodiments, the compositions comprise: i) leucine, from about 13% (wt) to about 15.9% (wt) lysine, phenylalanine, valine, threonine, isoleucine, methionine, histidine, and tryptophan; ii) at least one conditionally essential amino acid; iii) at least one conditionally essential amino acid precursor; and iv) at least one non-standard amino acid. In some embodiments, the conditionally essential amino acid is arginine or citrulline and the non-standard amino acid is L-carnitine.

Keto acids or ketoacids (also called oxo acids or oxoacids) are organic compounds that contain a carboxylic acid group and a ketone group. The keto acids are precursors of the amino acids in humans, specifically, valine derives from the animation of alpha-ketoisovaleric acid; phenylalanine derives from the amination of phenylpyruvic acid; methionine derives from the amination of alpha-keto-gamma-methylthiobutyric acid; leucine derives from the amination of alpha-ketoisocaproic acid; isoleucine derives from the amination of alpha-keto-beta-methylvaleric acid; histidine derives from the amination of imidazolepyruvic acid; tryptophan derives from the amination of indolepyruvic acid; lysine derives from the amination of alpha-keto-gamma-aminocaproic acid; and threonine derives from the amination of alpha-keto-beta-hydroxybutyric acid. Thus, one or more of the keto-analogs of the amino acids disclosed herein may be used in place of the amino acids.

The hydroxy-acid analogs of methionine, phenylalanine, and isoleucine can also be used in place of their respective amino acids.

In some embodiments, the compositions are liquids or solutions. In some embodiments, the compositions are solids or powder. In some embodiments, the composition further comprises an excipient. In some embodiments, the composition is provided orally, for example by liquid administration, chewable bite (U.S. Pat. No. 7,223,417), by capsule, or by tube.

In some embodiments, the compositions can also comprise a component for liquid administration. In some embodiments, the compositions can also comprise an excipient of supplemental minerals. Suitable minerals include, but are not limited to, one or more minerals or mineral sources. Examples of minerals include, but are not limited to: chloride, sodium, calcium, iron, chromium, copper, iodine, zinc, magnesium, manganese, molybdenum, phosphorus, potassium, and selenium, or any combination thereof. Suitable forms of any of the foregoing minerals include, but are not limited to, soluble mineral salts, slightly soluble mineral salts, insoluble mineral salts, chelated minerals, mineral complexes, non-reactive minerals such as carbonyl minerals, and reduced minerals, or any combination thereof.

In some embodiments, the compositions can also comprise one or more vitamins. The vitamins may be fat-soluble or water soluble vitamins. Suitable vitamins include, but are not limited to, vitamin C, vitamin A, vitamin E, vitamin B12, vitamin K, riboflavin, niacin, vitamin D, vitamin B6, folic acid, pyridoxine, thiamine, pantothenic acid, and biotin, or any combination thereof. The form of the vitamin can include a salt of the vitamin, a derivative of the vitamin, compounds having the same or similar activity of a vitamin, and a metabolite of a vitamin.

In some embodiments, one or more low glycemic carbohydrates can be added to the compositions to provide the energy produce proteins without eliciting a significant insulin response. This energy source may also drive muscle growth more efficiently than food or other protein supplements to achieve muscle synthesis without causing subjects to compensate for increased calorie intake. Such compounds, which may also be referred to as slow release saccharides, include, but are not limited to, complex carbohydrates with long carbon chain lengths, including, but not limited to, nutriose, sucramalt, isomaltulose, dextrans, maltodextrin, and their functional equivalents, or any combination thereof. The elderly are generally resistant to the action of insulin, so avoiding an insulin response by using low glycemic carbohydrate will be advantageous to that population.

In some embodiments, low glycemic carbohydrate may comprise about 0% to about 50% (wt). About two times the carbohydrate as total amino acids can be added, as this will provide the amount of energy needed for the stimulation of muscle protein synthesis. In some embodiments, up to 30 g of carbohydrate per serving can be added.

In some embodiments, the compositions can also comprise at least one excipient. Examples of suitable excipients include, but are not limited to, a buffering agent, a preservative, a stabilizer, a binder, a compaction agent, a lubricant, a dispersion enhancer, a disintegration agent, a flavoring agent, a sweetener, and a coloring agent, or any combination thereof.

In some embodiments, the excipient is a buffering agent. Examples of suitable buffering agents include, but are not limited to, sodium citrate, magnesium carbonate, magnesium bicarbonate, calcium carbonate, and calcium bicarbonate, or any combination thereof.

In some embodiments, the excipient is a preservative. Suitable examples of preservatives include, but are not limited to, antioxidants, such as alpha-tocopherol or ascorbate, and antimicrobials, such as parabens, chlorobutanol, and phenol, or any combination thereof.

In some embodiments, the excipient is a binder. Suitable binders include, but are not limited to, starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohols, polyethylene glycol, polyols, saccharides, oligosaccharides, polypeptides, and oligopeptides, or any combination thereof.

In some embodiments, the excipient is a lubricant. Suitable examples of lubricants include, but are not limited to, magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil, or any combination thereof.

In some embodiments, the excipient is a dispersion enhancer. Suitable dispersants include, but are not limited to, starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants, or any combination thereof.

In some embodiments, the excipient is a disintegrant. The disintegrant can be a non-effervescent disintegrant. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth, or any combination thereof. The disintegrant can be an effervescent disintegrant. Suitable effervescent disintegrants include, but are not limited to, sodium bicarbonate in combination with citric acid, and sodium bicarbonate in combination with tartaric acid, or any combination thereof.

In some embodiments, the excipient is a flavoring agent. Flavoring agents can be incorporated into an outer layer and can be chosen from synthetic flavor oils and flavoring aromatics; natural oils; extracts from plants, leaves, flowers, and fruits; or any combination thereof. Flavoring agents include, but are not limited to, cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot, or any combination thereof.

In some embodiments, the excipient is a sweetener. Sweeteners include, but are not limited to, glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia rebaudiana derivatives (Stevioside, rebaudioside A or C); chloro derivatives of sucrose such as sucralose; and sugar alcohols such as sorbitol, mannitol, xylitol, and the like, or any combination thereof. Also contemplated as sweeteners are hydrogenated starch hydrolysates and 3,6-dihydro-6-methyl-1,2,3-oxathiazin-4-one-2,2-dioxide, particularly the potassium salt (acesulfame-K), and sodium and calcium salts thereof.

In some embodiments, the compositions can also comprise a coloring agent. Suitable color additives include, but are not limited to, food, drug, and cosmetic colors (FD&C), drug and cosmetic colors (D&C), or external drug and cosmetic colors (Ext. D&C). These colors or dyes, along with their corresponding lakes, and certain natural and derived colorants, may be suitable for use herein.

In some embodiments, the weight fraction of the excipient or combination of excipients in the formulation can be from about 0.01% to about 30% of the total weight of the amino acid composition.

The compositions can be in a form suitable for oral use (for example as tablets, lozenges, hard or soft capsules, aqueous or oily suspensions, emulsions, dispersible powders or granules, syrups or elixirs, medical food products, nutraceuticals, or pastes), for topical use (for example as pastes, creams, ointments, gels, or aqueous or oily solutions or suspensions), for administration by inhalation (for example as finely divided powder) for parental administration (for example as a sterile aqueous or oily solution for intravenous, subcutaneous, intramuscular dosing or as a suppository for rectal dosing) or for enteral administration (for example via tube feeding).

Solid dosage forms for oral administration can include capsules, tablets, caplets, pills, troches, chewables, lozenges, powders, and granules. A capsule typically comprises a core material comprising a composition and a shell wall that encapsulates the core material. The core material can be solid, liquid, or an emulsion. The shell wall material may comprise soft gelatin, hard gelatin, or a polymer. Suitable polymers include, but are not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac), or any combination thereof. Some such polymers may also function as taste-masking agents.

Tablets, pills, and the like may be compressed, multiply compressed, multiply layered, and/or coated. The coating may be single or multiple. In some embodiments, the coating material may comprise a polysaccharide or a mixture of saccharides and glycoproteins extracted from a plant, fungus, or microbe. Examples include, but are not limited to, corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum, or any combination thereof. In some embodiments, the coating material may comprise a protein. Suitable proteins include, but are not limited to, gelatin, casein, collagen, whey proteins, soy proteins, rice protein, and corn proteins, or any combination thereof. In some embodiments, the coating material may comprise a fat or oil, and in particular, a high temperature melting fat or oil. The fat or oil may be hydrogenated or partially hydrogenated, and can be derived from a plant. The fat or oil may comprise glycerides, free fatty acids, fatty acid esters, or a mixture thereof. In some embodiments, the coating material may comprise an edible wax. Edible waxes may be derived from animals, insects, or plants. Examples include, but are not limited to, beeswax, lanolin, bayberry wax, carnauba wax, and rice bran wax. Tablets and pills may additionally be prepared with enteric coatings.

In some embodiments, the composition can be in the form of a powder or granule. In some embodiments, the compositions can be incorporated into a food product. In some embodiments, the food product may be a drink for oral administration. Examples of suitable drinks include, but are not limited to, fruit juice, a fruit drink, an artificially flavored drink, an artificially sweetened drink, a carbonated beverage, a sports drink, a liquid diary product, a shake, and so forth to which excipients may also be added. Other suitable means for oral administration include aqueous and nonaqueous solutions, emulsions, suspensions and solutions and/or suspensions reconstituted from non-effervescent granules.

The beverage products disclosed here optionally contain one or more flavor components, for example, natural or synthetic fruit flavors, botanical flavors, other flavors, or any mixtures thereof. Fruit flavors include flavors derived from the edible reproductive part of a seed plant, such as, banana, tomato, cranberry and the like, and those having a small, fleshy berry. Fruit flavors also include synthetically prepared flavors made to simulate fruit flavors derived from natural sources. Examples of suitable fruit or berry sources include, but are not limited to, whole berries or portions thereof, berry juice, berry juice concentrates, berry purees and blends thereof, dried berry powders, dried berry juice powders, and the like.

Exemplary fruit flavors include, but are not limited to, the citrus flavors, e.g., orange, lemon, lime and grapefruit, flavors such as apple, grape, cherry, and pineapple flavors and the like, or any mixture thereof. In some embodiments, the beverage products comprise a fruit flavor component, e.g., a juice concentrate or juice. Botanical flavors (i.e., flavors derived from parts of a plant other than the fruit) include botanical flavors such as, for example, flavors derived from essential oils and extracts of nuts, bark, roots and leaves, can be used herein. Botanical flavors also include synthetically prepared flavors made to simulate botanical flavors derived from natural sources. Examples of such flavors include, but are not limited to, cola flavors, tea flavors, and the like, or any mixture thereof. The flavor component can further comprise a blend of the various above-mentioned flavors. In some embodiments, a cola flavor component or a tea flavor component is used. The particular amount of the flavor component useful for imparting flavor characteristics to the beverages will depend upon the flavor(s) selected, the flavor impression desired, and the form of the flavor component.

Juices suitable include, but are not limited to, fruit, vegetable, and berry juices. Juices can be employed in the form of a concentrate, puree, single-strength juice, or other suitable forms. Juices include, but are not limited to, single-strength fruit, berry, or vegetable juice, as well as concentrates, purees, milks, and other forms. Multiple different fruit, vegetable and/or berry juices can be combined, optionally, along with other flavorings, to generate a beverage having a desired flavor. Examples of suitable juice sources include, but are not limited to, plum, prune, date, currant, fig, grape, raisin, cranberry, pineapple, peach, banana, apple, pear, guava, apricot, Saskatoon berry, blueberry, plains berry, prairie berry, mulberry, elderberry, Barbados cherry (acerola cherry), choke cherry, date, coconut, olive, raspberry, strawberry, huckleberry, loganberry, currant, dewberry, boysenberry, kiwi, cherry, blackberry, quince, buckthorn, passion fruit, sloe, rowan, gooseberry, pomegranate, persimmon, mango, rhubarb, papaya, litchi, lemon, orange, lime, tangerine, tangerine, mandarin orange, tangelo, pomelo, grapefruit, yumberry, etc. In the beverages employing juice, juice may be used, for example, at a level of at least about 0.2% by weight of the beverage. In some embodiments, juice is employed at a level of from about 0.2% to about 40% by weight of the beverage.

Organic acids compatible with the components may be employed in an embodiment. Such acids include citric acid, malic acid and other food compatible acids.

Certain such juices which are lighter in color can be included in the compositions to adjust the flavor and/or increase the juice content of the beverage without darkening the beverage color. Examples of such juices include, but are not limited to, apple, pear, pineapple, peach, lemon, lime, orange, apricot, grapefruit, tangerine, rhubarb, cassis, quince, passion fruit, papaya, mango, guava, litchi, kiwi, mandarin, coconut, and banana. Deflavored and decolored juices can be employed if desired.

The compositions may also be a solid foodstuff. Suitable examples of a solid foodstuff include, but are not limited to, a food bar, a snack bar, a cookie, a brownie, a muffin, a cracker, a chewable gum, an ice cream bar, a frozen yogurt bar, a chewy snack and the like.

The present disclosure also provides methods for attenuating muscle atrophy, restoring muscle function, enhancing improvement score, reducing M1 macrophage level, decreasing inflammation, or improving surgical recovery time in patients who have undergone a musculoskeletal surgery, or any combination thereof, by administering to the patient any of the compositions described herein. In some embodiments, the patient is about to undergo or has undergone a musculoskeletal surgery and the method of delivering may be oral, such as through liquid or edible administration.

The composition as described herein can be administered to improve (e.g., enhance) muscle function and performance in a patient with a muscle disease or disorder or who has undergone or will undergo a surgery. The present disclosure also provides methods for attenuating one, two, three, four, five, six, seven, eight, nine, or more (e.g., all) physiological symptoms selected from immobilization, malnutrition, fasting, aging, autophagy, reduced protein synthesis, anabolic resistance, neuromuscular junction integrity, insulin resistance, decreased mitochondrial biogenesis, anaplerosis, or an energy deficit. The methods include administering to a subject in need thereof an effective amount of the composition. In some embodiments, the subject has a rare muscle disease. In some embodiments, the subject has muscle atrophy, sarcopenia, muscle deterioration, muscle decay, cachexia, drug-induced myopathy, muscular dystrophy, or myopenia.

The phrases “improving muscle function”, “improving muscle performance” “enhancing muscle function” and, “enhancing muscle performance” refer to the desired biological response. In some embodiments, the desired biological response comprises one or more of a decrease/reduction in muscle atrophy, a decrease/reduction in the severity of muscle atrophy (such as, for example, a reduction or inhibition of muscle atrophy), a decrease/reduction in symptoms and muscle atrophy-related effects, delaying the onset of symptoms and muscle atrophy-related effects, reducing the severity of symptoms of muscle atrophy-related effects, reducing the severity of an acute episode, reducing the number of symptoms and muscle atrophy-related effects, reducing the latency of symptoms and muscle atrophy-related effects, an amelioration of symptoms and muscle atrophy-related effects, reducing secondary symptoms, reducing secondary infections, preventing relapse to muscle atrophy, decreasing the number or frequency of relapse episodes, increasing latency between symptomatic episodes, increasing time to sustained progression, speeding recovery, and/or increasing efficacy of or decreasing resistance to alternative therapeutics, following administration of the composition. In some embodiments, the desired biological response comprises one or more of a complete or partial avoidance/inhibition or a delay of muscle atrophy development/progression (such as, for example, a complete or partial avoidance/inhibition or a delay) following administration of a composition. In some embodiments, muscle atrophy can be any form of muscle atrophy at any clinical stage or manifestation, the delay of the onset or evolution or aggravation or deterioration of the symptoms or signs of muscle atrophy, and/or preventing and/or reducing the severity of muscle atrophy. In some embodiments, the desired biological response comprises one or more of attenuation of muscle atrophy, amelioration of muscle atrophy, eliminating of muscle atrophy, reversing muscle atrophy, or preventing of muscle atrophy. In some embodiments, the desired biological response comprises preserving muscle function, restoring the muscle function, attenuating the loss of muscle function, ameliorating the loss of muscle function, or reversing the loss of muscle function. In some embodiments, the desired biological response comprises enhancing muscle strength, preserving muscle strength, attenuating the loss of muscle strength, ameliorating the loss of muscle strength, or reversing the loss of muscle strength. In some embodiments, the desired biological response comprises enhancing muscle volume, preserving muscle volume, attenuating the loss of muscle volume, ameliorating the loss of muscle volume, or reversing the loss of muscle volume. In some embodiments, the desired biological response comprises enhancing wound healing.

In some embodiments, the subject has a muscle disease or disorder. In some embodiments, the muscle disease or disorder is a dystrophy. In some embodiments, the muscle disease or disorder is a myotonic dystrophy. In some embodiments, the muscle disease or disorder is DM1. In some embodiments, the subject has muscle atrophy, sarcopenia, muscle deterioration, muscle decay, cachexia, drug-induced myopathy, muscular dystrophy, or myopenia.

In some embodiments, the muscle disease or disorder is a drug-induced myopathy. In some embodiments, the muscle disease or disorder is a statin-induced myopathy. In some embodiments, the muscle disease or disorder is a steroid-induced myopathy. In some embodiments, the muscle disease or disorder is an immunosuppressant-induced myopathy. In some embodiments, the muscle disease or disorder is a chemotherapeutic-induced myopathy. In some embodiments, the muscle disease or disorder is an alcohol-induced myopathy.

In some embodiments, the subject has a fracture or other trauma. In some embodiments, the method includes administering to a subject in need thereof an effective amount of the composition to treat immobilization. In some embodiments, the method includes administering to a subject in need thereof an effective amount of the composition to attenuate or ameliorate malnutrition. In some embodiments, the method includes administering to a subject in need thereof an effective amount of the composition to attenuate or ameliorate fasting. In some embodiments, the method includes administering to a subject in need thereof an effective amount of the composition to attenuate or ameliorate aging. In some embodiments, the method includes administering to a subject in need thereof an effective amount of the composition to attenuate or ameliorate autophagy. In some embodiments, the method includes administering to a subject in need thereof an effective amount of the composition to attenuate or ameliorate reduced protein synthesis. In some embodiments, the method includes administering to a subject in need thereof an effective amount of the composition to attenuate or ameliorate anabolic resistance. In some embodiments, the method includes administering to a subject in need thereof an effective amount of the composition to preserve junction integrity (e.g., neuromuscular junction integrity). In some embodiments, the method includes administering to a subject in need thereof an effective amount of the composition to attenuate or ameliorate insulin resistance. In some embodiments, the method includes administering to a subject in need thereof an effective amount of the composition to attenuate or ameliorate decreased mitochondrial biogenesis. In some embodiments, the method includes administering to a subject in need thereof an effective amount of the composition to attenuate or ameliorate anaplerosis. In some embodiments, the method includes administering to a subject in need thereof an effective amount of the composition to attenuate or ameliorate an energy deficit.

The present disclosure also provides methods for enhancing or restoring muscle function that include administering to a subject in need thereof an effective amount of any of the compositions described herein. In some embodiments, the subject has or is identified as having decreased muscle function due to aging, injury, atrophy, infection, or disease. In some embodiments, the composition reduces muscle atrophy in the subject. In some embodiments, the composition maintains muscle volume in the subject. In some embodiments, the composition attenuates muscle atrophy in the subject. In some embodiments, the composition restores functional strength and mobility in the subject. In some embodiments, the composition improves wound healing in the subject. In some embodiments, the composition enhances the improvement scores and patient outcomes. In some embodiments, the composition accelerates subject's recovery, for example from musculoskeletal injury.

In some embodiments, the subject has or is identified as having muscle deterioration, decay, atrophy, cachexia, sarcopenia, drug-induced myopathy, muscular dystrophy, or myopenia. In some embodiments, the subject has or is identified as having muscle deterioration. In some embodiments, the subject has or is identified as having muscle decay. In some embodiments, the subject has or is identified as having muscle atrophy. In some embodiments, the subject has or is identified as having cachexia. In some embodiments, the subject has or is identified as having sarcopenia. In some embodiments, the subject has or is identified as having drug-induced myopathy. In some embodiments, the subject has or is identified as having muscular dystrophy. In some embodiments, the subject has or is identified as having myopenia. In some embodiments, the subject is a human. In some embodiments, the subject has not received prior treatment with a composition including defined amino acid components (e.g., a naive subject).

In some embodiments, the subject has muscle weakness, e.g., muscle weakness of one, two, three, or more (e.g., all) of skeletal muscle, cardiac muscle, or smooth muscle. In some embodiments, the subject has muscle weakness in one, two, three, four, five, six, or more (e.g., all) of a neck muscle, a torso muscle, an arm muscle, a shoulder muscle, a hand muscle, a leg muscle, or a foot muscle.

In some embodiments, the subject has had a musculoskeletal surgery or has worn a cast prior to administration of the composition. Musculoskeletal surgeries include, but are not limited to: an arthroplasty, including, but not limited to, total knee arthroplasty and total hip arthroplasty; reconstruction of a ligament, such as the anterior cruciate ligament; a carpal tunnel surgery; a rotator cuff tendon repair; a lumbar spinal fusion; knee surgery; hip surgery; or an ankle fracture repair. In some embodiments, the subject has had rotator cuff surgery prior to administration of the composition. In some embodiments, the subject has had a knee surgery prior to administration of the composition. In some embodiments, the subject has had a total knee arthroplasty prior to administration of the composition. In some embodiments, the subject has had hip surgery prior to administration of the composition. In some embodiments, the subject has had total hip arthroplasty prior to administration of the composition. In some embodiments, the subject has had an injury repair surgery. In some embodiments, the subject has worn a cast prior to administration of the composition.

In some embodiments, the subject has perceived muscle weakness, e.g., chronic fatigue syndrome. In some embodiments, the subject has a cancer-associated muscle weakness. In some embodiments, the subject has a neuromuscular disorder, e.g., myasthenia gravis or Lambert-Eaton myasthenic syndrome. In some embodiments, the subject has muscular dystrophy, e.g., Duchenne muscular dystrophy, Becker muscular dystrophy, facioscapulohumeral muscular dystrophy, or myotonic dystrophy. In some embodiments, the subject has inflammatory myopathy, e.g., polymyositis or dermatomyositis.

In some embodiments, the subject has one, two, or more (e.g., all) of low sodium levels (e.g., hyponatremia), low potassium levels (e.g., hypokalemia), or a calcium deficiency or relatively high calcium levels (e.g., hypercalcemia).

In some embodiments, the subject has muscle weakness associated with nerve damage, e.g., neuralgia or peripheral neuropathy. In some embodiments, the subject has a bone weakness disease, e.g., osteomalacia, osteogenesis imperfecta, rickets, or Hypophosphatasia.

In some embodiments, the subject has experienced a stroke or a transient ischemic attack. In some embodiments, the subject has an autoimmune disease, e.g., Graves' disease.

In some embodiments, the subject has hypothyroidism. In some embodiments, the subject has amyotrophic lateral sclerosis (ALS). In some embodiments, administering the composition results in an improvement in one or more metabolic symptoms in the subject. In some embodiments, the one or more metabolic symptoms is selected from the following: mTORC1 activation; improved insulin sensitivity; activation of muscle protein synthesis; scavenging of reactive oxygen species (ROS); decreased inflammation; inhibition catabolism; ammonia detoxification; and decreased fibrosis progression.

In some embodiments, the composition reduces muscle atrophy in the subject. In some embodiments, the composition attenuates muscle atrophy in the subject. In some embodiments, the composition ameliorates muscle atrophy in the subject. In some embodiments, the composition eliminates muscle atrophy in the subject. In some embodiments, the composition reverses muscle atrophy in the subject. In some embodiments, the composition prevents muscle atrophy in the subject.

In some embodiments, the composition preserves muscle function in the subject. In some embodiments, the composition restores the muscle function in the subject. In some embodiments, the composition attenuates the loss of muscle function in the subject. In some embodiments, the composition ameliorates the loss of muscle function in the subject. In some embodiments, the composition reverses the loss of muscle function in the subject.

In some embodiments, the composition enhances muscle strength in the subject. In some embodiments, the composition preserves muscle strength in the subject. In some embodiments, the composition attenuates the loss of muscle strength in the subject. In some embodiments, the composition ameliorates the loss of muscle strength in the subject. In some embodiments, the composition reverses the loss of muscle strength in the subject.

In some embodiments, the composition enhances muscle volume in the subject. In some embodiments, the composition preserves muscle volume in the subject. In some embodiments, the composition attenuates the loss of muscle volume in the subject. In some embodiments, the composition ameliorates the loss of muscle volume in the subject. In some embodiments, the composition reverses the loss of muscle volume in the subject.

In some embodiments, the composition enhances wound healing in the subject.

In some embodiments, the composition restores functional strength and mobility in the subject. In some embodiments, the composition enhances the improvement scores and patient outcomes.

In some embodiments, the composition results in enhanced anabolism and catabolism of muscle tissue in the subject.

In some embodiments, administering the composition results in activation of muscle protein synthesis in the subject, decreased inflammation in the subject, inhibited catabolism in the subject, ammonia detoxification in the subject, and/or decreased fibrosis progression in the subject. In some embodiments, the composition also reduces muscle atrophy. In some embodiments, the composition attenuates muscle atrophy in the subject. In some embodiments, the composition restores functional strength and mobility in the subject. In some embodiments, the composition maintains muscle volume in the subject. In some embodiments, the composition enhances the improvement scores and patient outcomes.

In some embodiments, the composition results in an improvement in one or both of muscle loss or muscle function related to one or both of immobilization or muscle disuse following injury in a subject. In some embodiments, the subject has had a musculoskeletal surgery or has worn a cast prior to administration of the composition.

In some embodiments, the composition results in reduction of M1 macrophage levels and decreased inflammation.

In some embodiments, the subject has ventilator-induced diaphragmatic dystrophy or ventilator-induced diaphragmatic dysfunction. In some embodiments, the subject has had one or both of ICU-acquired or burns-related myopathies.

In some embodiments, the subject has disease-related cachexia, e.g., a disease-related cachexia selected from chronic obstructive pulmonary disease (COPD), congestive heart failure (CHF), chronic kidney disease (CKD), and cancer.

In some embodiments, the composition is administered with a second agent.

The present disclosure also provides methods for reducing muscle atrophy comprising administering to a subject in need thereof an effective amount of a composition described herein.

The present disclosure also provides compositions described herein for use as a medicament. The present disclosure also provides a composition described herein for use as a nutritional supplement.

The present disclosure provides compositions described herein for use as a medicament in enhancing muscle function. The present disclosure also provides compositions described herein for use as a nutritional supplement in enhancing muscle function.

The present disclosure provides compositions described herein for use as a medicament for treating one or more symptoms selected from the group consisting of immobilization, malnutrition, fasting, aging, autophagy, reduced protein synthesis, anabolic resistance, neuromuscular junction integrity, insulin resistance, decreased mitochondrial biogenesis, anaplerosis, and atrophy.

The present disclosure provides compositions described herein for use as a nutritional supplement for attenuating or ameliorating one or more symptoms selected from the group consisting of immobilization, malnutrition, fasting, aging, autophagy, reduced protein synthesis, anabolic resistance, neuromuscular junction integrity, insulin resistance, decreased mitochondrial biogenesis, anaplerosis, and atrophy.

The present disclosure also provides compositions described herein for use in the manufacture of a medicament for enhancing muscle function. The present disclosure provides compositions described herein for use in the manufacture of a nutritional supplement for enhancing muscle function. The present disclosure provides uses of a composition for the manufacture of a medicament for treating one or more symptoms selected from the group consisting of immobilization, malnutrition, fasting, aging, autophagy, reduced protein synthesis, anabolic resistance, neuromuscular junction integrity, insulin resistance, decreased mitochondrial biogenesis, anaplerosis, and atrophy. The present disclosure provides uses of a composition for the manufacture of a nutritional supplement for attenuating or ameliorating one or more symptoms selected from the group consisting of immobilization, malnutrition, fasting, aging, autophagy, reduced protein synthesis, anabolic resistance, neuromuscular junction integrity, insulin resistance, decreased mitochondrial biogenesis, anaplerosis, and atrophy.

The composition can be administered according to a dosage regimen described herein to, e.g., enhance muscle function in a subject (e.g., a human, such as a human with muscle atrophy). The composition can be administered according to a dosage regimen described herein to treat (e.g., inhibit, reduce, or prevent) a disorder, e.g., a muscle disease in a subject (e.g., a human).

In some embodiments, the composition can be provided to a patient in either a single or multiple dosage regimens. In some embodiments, doses can be administered, e.g., twice daily, three times daily, four times daily, five times daily, six times daily, seven times daily, or more. In some embodiments, the composition is administered for at least 2 days, 3 days, 4 days, 5 days, 6 days, 7 days, or 2 weeks. In some embodiments, the composition is administered for at least 10 weeks, 11 weeks, 12 weeks, 13 weeks, 14 weeks, 15 weeks, 16 weeks, 17 weeks, 18 weeks, 19 weeks, 20 weeks, or longer. In some embodiments, the composition can be administered chronically, e.g., more than 30 days, e.g., 31 days, 40 days, 50 days, 60 days, 3 months, 6 months, 9 months, one year, two years, or three years).

In some embodiments, the composition is administered at a dose of about 4 g to about 80 g total amino acids, e.g., once per day, twice per day, three times per day, four times per day, five times per day, or six times per day (e.g., three times per day). In some embodiments, the composition is administered at a dose of about 5 g to about 15 g, about 10 g to about 20 g, about 20 g to about 40 g, or about 25 g to about 50 g total amino acids, e.g., once per day, twice per day, three times per day, four times per day, five times per day, or six times per day (e.g., three times per day).

In some embodiments, the composition is administered at a dose of about 25 g to about 50 g (e.g., about 23.4 to 46.8 g total amino acids), e.g., once per day, twice per day, three times per day, four times per day, five times per day, or six times per day (e.g., three times per day). In some embodiments, about 50 g total amino acids is administered per day.

As will be appreciated by one of skill in the art, the dose (also referenced herein as serving amount or amount administered, e.g., in a single administration) of the composition can vary depending on the body weight, sex, age and/or medical condition of the subject, the intensity of the physical exercise, the length of bed rest, the severity of the muscle damage or trauma suffered by the subject, the method of administration, and the duration of rehabilitation. The dosage of the compositions is from about 20 to about 1000 mg/kg of body weight/day, or from about 40 to about 350 mg/kg of body weight (23.4 to 46.8 g/day for an average size person (154 lb) is typical). Typical doses of the composition for oral administration may be about 1.5 g per dose. In some embodiments, a dose of about 1, 2, 2.5, 3, 3.5, 5, 4, 4.5, 5, or 5.5 g of the composition may be administered. In some embodiments, a dose of about 6, 7, 8, 9, 10, 11, 12, 13, 14, 15, 16, 17, 18, 19 or 20 g of the composition may be administered. In some embodiments, a dose of about 20, 21, 22, 23, 24, 25, 26, 27, 28, 29, 30, 31, 32, 33, 34, 35, 36, 37, 38, 39, 40, 41, 42 43, 44, 45, 46 or 47 g of the composition may be administered each day.

In some embodiments, the composition dose may be about 23.4 g per dose, and comprise about 7400 mg of L-leucine, about 3600 mg of L-lysine, about 3100 mg of L-phenylalanine, about 2080 of L-valine, about 1900 mg of L-threonine, about 1800 L-isoleucine, about 1760 of L-methionine, about 1280 mg of L-histidine, and about 500 mg of L-tryptophan.

Administering multiple doses of the composition per day may also be used as needed to provide the desired level of recovery of muscle strength and function. For instance, one, two, three, four, or more doses of the composition may be administered per day. In some embodiments, one dose of the composition may be administered per day. in another preferred embodiment, two doses of the composition may be administered per day. In some embodiments, three doses of the composition may be administered per day.

In some embodiments, the composition is administered every 2 hours, every 3 hours, every 4 hours, every 5 hours, every 6 hours, every 7 hours, every 8 hours, every 9 hours, or every 10 hours.

In some embodiments, the composition is administered prior to a meal (e.g., one, two, or more (e.g., all) of breakfast, lunch, or dinner). In some embodiments, the composition is administered concurrent with a meal (e.g., one, two, or more (e.g., all) of breakfast, lunch, or dinner). In some embodiments, the composition is administered following a meal (e.g., one, two, or more (e.g., all) of breakfast, lunch, or dinner).

The timing and duration of administration of the composition can vary. For instance, when the composition is administered to a subject that has undergone a musculoskeletal surgery, the composition may be administered exclusively before surgery, exclusively after surgery, or both before and after surgery. In some embodiments, the composition is administered before surgery, starting 1 week prior to surgery. In some embodiments, the composition is administered before surgery, starting 2 weeks prior to surgery. In some embodiments, the composition is administered before surgery, starting 3 weeks prior to surgery. In some embodiments, the composition is administered before surgery, starting 4 weeks prior to surgery. In some embodiments, the composition is administered before surgery, starting 5 weeks prior to surgery. In some embodiments, the composition is administered before surgery, starting 6 weeks prior to surgery. In some embodiments, the composition is administered before surgery, starting 7 weeks prior to surgery. In some embodiments, the composition is administered before surgery, starting 8 weeks prior to surgery. In some embodiments, the composition is administered before surgery, starting 9 weeks prior to surgery. In some embodiments, the composition is administered before surgery, starting 10 weeks prior to surgery. In some embodiments, the composition is administered before surgery, starting 11 weeks prior to surgery. In some embodiments, the composition is administered before surgery, starting 12 weeks prior to surgery.

In some embodiments, administration is maintained after surgery, continuing for 1 week following surgery. In some embodiments, administration is maintained after surgery, continuing for 2 weeks following surgery. In some embodiments, administration is maintained after surgery, continuing for 3 weeks following surgery. In some embodiments, administration is maintained after surgery, continuing for 4 weeks following surgery. In some embodiments, administration is maintained after surgery, continuing for 5 weeks following surgery. In some embodiments, administration is maintained after surgery, continuing for 6 weeks following surgery. In some embodiments, administration is maintained after surgery, continuing for 7 weeks following surgery. In some embodiments, administration is maintained after surgery, continuing for 8 weeks following surgery. In some embodiments, administration is maintained after surgery, continuing for 9 weeks following surgery. In some embodiments, administration is maintained after surgery, continuing for 10 weeks following surgery. In some embodiments, administration is maintained after surgery, continuing for 11 weeks following surgery. In some embodiments, administration is maintained after surgery, continuing for 12 weeks following surgery.

In some embodiments, a daily regimen of two doses per day is initiated two weeks prior to surgery and is maintained for two weeks following the surgery. In some embodiments, the composition regimen is maintained until full restoration of muscle function and strength is attained.

In some embodiments, the compositions can be administered to a subject orally. In some embodiments, the compositions can be administered to a subject by any suitable route including, but not limited to, by injection intravenously, subcutaneously, intraperitoneally, intramuscularly, intramedullary, intraventricularly, intraepidurally, intraarterially, intravascularly, intraarticularly, intrasynovially, intrasternally, intrathecally, intrahepatically, intraspinally, intratumorly, intracranially, enteral, intrapulmonary, transmucosal, intrauterine, sublingual, or locally at sites of injury or surgery. In some embodiments, the compositions can be administered to a subject by routes including nasal, ophthalmic, rectal, or topical. In some embodiments, compositions are administered as a sustained release composition or device, such as a Medipad™ device. The composition can also be administered via the respiratory tract, for example, using a dry powder inhalation device, nebulizer, or a metered dose inhaler. The composition can also be administered by traditional syringes, needleless injection devices, “microprojectile bombardment gone guns,” or other physical methods such as electroporation (“EP”), “hydrodynamic method”, or ultrasound.

In some embodiments, the composition can be administered to a subject by sustained release administration, by such means as depot injections of erodible implants directly applied during surgery or by implantation of an infusion pump or a biocompatible sustained release implant into the subject. In some embodiments, the composition can be administered to a subject by injectable depot routes of administration, such as by using 1-, 3-, or 6-month depot injectable or biodegradable materials and methods, or by applying to the skin of the subject a transdermal patch containing the composition, and leaving the patch in contact with the subject's skin, generally for 1 to 5 hours per patch.

In order that the subject matter disclosed herein may be more efficiently understood, examples are provided below. It should be understood that these examples are for illustrative purposes only and are not to be construed as limiting the claimed subject matter in any manner.

### EXAMPLES

### Example 1: Materials and Methods

### Study Overview

In a single-center, 2-arm, double-blind, placebo-controlled, parallel-group study, patients will be allocated to treatment with EAA or placebo in a 1:1 ratio. Subjects ranging from about 50 to 80 years of age under the care of a physician will be participating.

Patients between 50 and 80 years of age scheduled to undergo primary unilateral TKA will be included. Exclusion criteria comprise a history of: lower extremity total joint replacement surgery; uncontrolled endocrine disease; heart, kidney, liver, blood, or respiratory disease; peripheral vascular disease; active cancer; and recent treatment with anabolic steroids or oral corticosteroids for >1 week. Patients will be stratified by sex and randomly assigned, with the optional use of a table generated with RANDOM.ORG, to ingest 20 g of EAA or placebo twice daily at about 10 a.m. and about 2 p.m. for about 7 days pre-operatively. Patients will then fast overnight, and surgery will be performed the following day. Supplementation with EAA or placebo will resume on the first post-operative day. Supplementation with EAA or placebo will continue for about 42 days after TKA surgery. Each patient will be issued a log book in which to record the time of supplement ingestion. Log book recordings will be verified by research and nursing staff during the inpatient stay and by research staff at each visit.

Preoperative blood chemistry, magnetic resonance imaging (MRI), strength and functional mobility, and patient-reported outcomes data will be collected at baseline (2 to 6 weeks pre-operatively). Blood chemistry will be monitored on the day of surgery, 2 days post-operatively, and 2 and 6 weeks post-operatively. Functional measures will be repeated at 2 and 6 weeks post-operatively. MRI assessment and evaluation of patient-reported outcomes will be performed at 6 weeks post-operatively. Patients will record physical activity via accelerometry and caloric intake with 3-day food diaries.

Although patient-reported outcomes, functional mobility, and muscle strength are examined, the present study is designed with sufficient statistical power to detect, primarily, differences in the more-reliable physiological measurements of muscle volume.

### Investigational Supplementation

Placebo or EAA (Table 1) supplementation will be initiated about 1 week before TKA, discontinued on the day of surgery, resumed on the first post-operative day, and continued for about 6 weeks, for a total of about 49 days. The supplement will be mixed into a liquid or a food and ingested twice daily between meals. During hospitalization, the supplement will be consumed about 1 hour after both morning and afternoon physical therapy. After discharge, patients will continue this regimen to maximize the anabolic/anticatabolic effect of EAA. Ingestion will be recorded and verified by research personnel. Supplements will be are prepared by a compounding organization with use of amino acids commercially purchased. Amino acids will be added in predefined proportions to achieve a master mix weighing about 23.42 g. The master mix will be vigorously shaken for several minutes and then aliquoted into 25 g vials and dispensed in blinded vials by study personnel.

TABLE 1
Supplement Composition

| | |
|---|---|
| Percentage of Total | Grams |

EAA

| | | |
|---|---|---|
| Histidine | 5.5% | 1.28 |
| Isoleucine | 7.7% | 1.8 |
| Leucine | 31.6% | 7.4 |
| Lysine | 15.4% | 3.6 |
| Methionine | 7.5% | 1.76 |
| Phenylalanine | 13.2% | 3.1 |
| Threonine | 8.1% | 1.9 |
| Valine | 8.9% | 2.08 |
| Tryptophan | 2.1% | 0.5 |
| Total | 100% | 23.42 |

Placebo

| | | |
|---|---|---|
| Alanine | 100% | 23.42 |

### Muscle Volume (Primary End Point)

MRI of both lower extremities will be performed. One researcher will analyze blinded MRI data.

### Secondary End Points

Software, such as Medical Image Processing, Analysis, and Visualization (MIPAV) software (v. 7.4.0, see, world wide web at “mipay.cit.nih.gov”), may be used to calculate mid-thigh muscle volume after excluding bone and isolating each muscle group. Magnetic eddy currents will be corrected with a nonparametric, nonuniform intensity normalization algorithm followed by fuzzy c-means adjustment.

Functional mobility measurements include handgrip strength, Short Physical Performance Battery (SPPB) balance (4-m gait speed and chair stand), timed up-and-go (TUG) test, 4 m walk, timed stair ascent and timed stair descent, leg strength, and 6 minute walk. The test order will be the same for all subjects.

Patients will wear an accelerometer for about 21 consecutive days from about 1 week pre-operatively through about 2 weeks post-operatively, as well as at about 5 and about 6 weeks post-operatively. Three-day food diaries on standardized days will be completed before surgery, about 1 and about 2 days post-operatively, and about 1, about 2, and about 5 weeks post-operatively. Software, such as Food Processor Nutrition Analysis Software (ESHA Research), will be used to calculate micronutrients and macronutrients.

At baseline and about 6 weeks post-operatively, patients will complete a survey, such as the Knee injury and Osteoarthritis Outcome Score (KOOS) survey, which has adequate construct validity and can demonstrate responsiveness to change, and the validated Veterans RAND 12-Item Health Survey (VR-12), which can yield Physical Component Summary (PCS12) and Mental Component Summary (MCS12) scores reflecting health-related quality of life. The previously validated Patient Health Questionnaire depression module (PHQ-9) may also be used, and measures depression symptom severity.

Fifteen blood tests will be used to monitor blood urea nitrogen, creatinine, creatinine clearance, homocysteine, and renal and liver function. Values at baseline, on the day of surgery, about 2 days post-operatively, and about 2 and about 6 weeks post-operatively will be reported for both treatment groups. An independent review, such as the National Institutes of Health-appointed Data and Safety Monitoring Board, will review adverse events and laboratory assessments. Nuclear Magnetic Resonance (NMR) will be used to randomly evaluate the EAA supplement composition.

### Statistical Analyses

Separate analysis models will be used to compare the EAA and placebo groups with regard to outcome variables at about 2 and about 6 weeks while controlling baseline values. A procedure, such as the Benjamini-Hochberg procedure, will be used to adjust p values for multiple comparisons.

### Example 2: Post Surgery Macrophage Level Reduction

M1 macrophage levels were monitored in patients who had undergone musculoskeletal surgery. An increase in M1 macrophage levels was observed post-surgery in the patients who received placebo (as compared to pre-surgery levels). In contrast, the patients who received the EAA composition (see, Table 1) displayed a decrease in M1 macrophage levels post-surgery (as compared to pre-surgery levels).

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A composition comprising: about 30% (wt) to about 34% (wt) L-leucine, or a salt thereof; about 13% (wt) to about 17% (wt) L-lysine, or a salt thereof; about 11% (wt) to about 15% (wt) L-phenylalanine, or a salt thereof; about 7% (wt) to about 11% (wt) L-valine, or a salt thereof; about 6% (wt) to about 10% (wt) L-threonine, or a salt thereof; about 6% (wt) to about 10% (wt) L-isoleucine, or a salt thereof; about 6% (wt) to about 10% (wt) L-methionine, or a salt thereof; about 3% (wt) to about 7% (wt) L-histidine, or a salt thereof; and about 1% (wt) to about 4% (wt) L-tryptophan, or a salt thereof.

2. The composition according to claim 1, comprising: about 31% (wt) to about 32% (wt) L-leucine, or a salt thereof; about 15% (wt) to about 16% (wt) L-lysine, or a salt thereof; about 13% (wt) to about 14% (wt) L-phenylalanine, or a salt thereof; about 8% (wt) to about 9% (wt) L-valine, or a salt thereof; about 8% (wt) to about 9% (wt) L-threonine, or a salt thereof; about 7% (wt) to about 8% (wt) L-isoleucine, or a salt thereof; about 7% (wt) to about 8% (wt) L-methionine, or a salt thereof; about 5% (wt) to about 6% (wt) L-histidine, or a salt thereof; and about 2% (wt) to about 3% (wt) L-tryptophan, or a salt thereof.

3. The composition according to claim 1, comprising: about 31.6% (wt) L-leucine, about 15.4% (wt) L-lysine, about 13.2% (wt) L-phenylalanine, about 8.9% (wt) L-valine, about 8.1% (wt) L-threonine, about 7.7% (wt) L-isoleucine, about 7.5% (wt) L-methionine, about 5.5% (wt) L-histidine, and about 2.1% (wt) L-tryptophan.

4. The composition according to claim 1, wherein the composition is in the form of a single serving comprising: about 7400 mg of L-leucine, about 3600 mg of L-lysine, about 3100 mg of L-phenylalanine, about 2080 mg of L-valine, about 1900 mg of L-threonine, about 1800 mg of L-isoleucine, about 1760 mg of L-methionine, about 1280 mg of L-histidine, and about 500 mg of L-tryptophan.

5. The composition according to claim 1, further comprising:
L-citrulline, or a salt thereof; L-cysteine, or a salt thereof; L-camitine, or a salt thereof; or creatine, or a salt thereof; or any mixture thereof.

6. The composition according to claim 1, wherein the composition is a powder.

7. The composition according to claim 1, wherein the composition is a solution.

8. The method of enhancing muscle performance in a human subject, the method comprising administering to the subject at least one serving of a composition comprising: about 30% (wt) to about 34% (wt) L-leucine, or a salt thereof; about 13% (wt) to about 17% (wt) L-lysine, or a salt thereof; about 11% (wt) to about 15% (wt) L-phenylalanine, or a salt thereof; about 7% (wt) to about 11% (wt) L-valine, or a salt thereof; about 6% (wt) to about 10% (wt) L-threonine, or a salt thereof; about 6% (wt) to about 10% (wt) L-isoleucine, or a salt thereof; about 6% (wt) to about 10% (wt) L-methionine, or a salt thereof; about 3% (wt) to about 7% (wt) L-histidine, or a salt thereof; and about 1% (wt) to about 4% (wt) L-tryptophan, or a salt thereof.

9. The method according to claim 8, wherein the composition comprises: about 31% (wt) to about 32% (wt) L-leucine, or a salt thereof; about 15% (wt) to about 16% (wt) L-lysine, or a salt thereof; about 13% (wt) to about 14% (wt) L-phenylalanine, or a salt thereof; about 8% (wt) to about 9% (wt) L-valine, or a salt thereof; about 8% (wt) to about 9% (wt) L-threonine, or a salt thereof; about 7% (wt) to about 8% (wt) L-isoleucine, or a salt thereof; about 7% (wt) to about 8% (wt) L-methionine, or a salt thereof; about 5% (wt) to about 6% (wt) L-histidine, or a salt thereof; and about 2% (wt) to about 3% (wt) L-tryptophan, or a salt thereof.

10. The method according to claim 8, wherein the composition comprises about 31.6% (wt) L-leucine, about 15.4% (wt) L-lysine, about 13.2% (wt) L-phenylalanine, about 8.9% (wt) L-valine, about 8.1% (wt) L-threonine, about 7.7% (wt) L-isoleucine, about 7.5% (wt) L-methionine, about 5.5% (wt) L-histidine, and about 2.1% (wt) L-tryptophan.

11. The method according to claim 8, wherein the composition is in the form of a single serving comprising about 7400 mg of L-leucine, about 3600 mg of L-lysine, about 3100 mg of L-phenylalanine, about 2080 mg of L-valine, about 1900 mg of L-threonine, about 1800 mg of L-isoleucine, about 1760 mg of L-methionine, about 1280 mg of L-histidine, and about 500 mg of L-tryptophan.

12. The method according to claim 8, wherein the composition further comprises: L-citrulline, or a salt thereof; L-cysteine, or a salt thereof; L-camitine, or a salt thereof; or creatine, or a salt thereof; or any mixture thereof.

13. The method according to claim 8, wherein the composition is a powder.

14. The method according to claim 8, wherein the composition is a solution.

15. The method according to claim 8, wherein a serving of the composition is administered at least twice a day.

16. The method according to claim 8, wherein the subject has undergone a musculoskeletal surgery.

17. The method according to claim 16, wherein the musculoskeletal surgery is an arthroplasty, reconstruction of a ligament, a carpal tunnel surgery, a rotator cuff tendon repair, a lumbar spinal fusion, or an ankle fracture repair.

18. The method according to claim 17, wherein the arthroplasty is a total knee arthroplasty or a total hip arthroplasty.

19. The method according to claim 16, wherein administration of the composition is initiated at least one week before surgery and continued for at least two weeks after surgery.

20. The method according to claim 16, wherein the composition is administered orally.
